(19) United States
(12) Patent Application Publication
(10) Pub. No.: US 2021/0272043 A1
(43) Pub. Date: Sep. 2, 2021

(54) PLACEMENT PLATFORM WITH MATCHING

(71) Applicant: SJ MedConnect, Inc., Santa Clara, CA (US)

(72) Inventors: Jason Reminick, Santa Clara, CA (US); Suzanne Karan, Rochester, NY (US)

(21) Appl. No.: 16/804,852

(22) Filed: Feb. 28, 2020

(51) Int. Cl. G06Q 10/06; G06Q 30/02

(52) U.S. Cl. CPC G06Q 10/063112; G06Q 30/0203; G06Q 10/06312; G06Q 10/063116

(57) ABSTRACT

Program placement can include: generating a user interface including at least one match indicator of how well a current user of a placement platform matches to one or more of a plurality of programs registered on the placement platform; and determining the match indicator by matching a set current data pertaining to how the current user has used the placement platform to seek placement among the programs to a set of history data pertaining to how each of a set of prior users of the placement platform had used the placement platform to seek placement among the programs.

### BACKGROUND

A placement platform can enable candidates seeking placement among highly sought-after limited-capacity programs to research and communicate with those programs, arrange interviews, etc. For example, a placement platform can enable graduating medical students to arrange interviews for placement among a variety of medical residency programs.

A placement platform can enable a candidate seeking placement to browse information about each available program, e.g., location, program highlights, types of candidates sought, etc. A placement platform can enable administrators of available programs to view profile information for candidates, e.g., location and demographic information, relevant test scores, etc.

### SUMMARY

In general, in one aspect, the invention relates to a placement platform with matching. A placement platform according to the invention can include: a user interface including at least one match indicator of how well a current user of the placement platform matches to one or more of a plurality of programs registered on the placement platform; and a data matcher that determines the match indicator by matching a set current data pertaining to how the current user has used the placement platform to seek placement among the programs to a set of history data pertaining to how each of a set of prior users of the placement platform had used the placement platform to seek placement among the programs.

In general, in another aspect, the invention relates to a method for program placement. The method can include: generating a user interface including at least one match indicator of how well a current user of a placement platform matches to one or more of a plurality of programs registered on the placement platform; and determining the match indicator by matching a set current data pertaining to how the current user has used the placement platform to seek placement among the programs to a set of history data pertaining to how each of a set of prior users of the placement platform had used the placement platform to seek placement among the programs.

Other aspects of the invention will be apparent from the following description and the appended claims.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 illustrates a placement platform with matching in one or more embodiments.

FIG. 2A illustrates an example of a user interface with a match indicator presented to a current user while the current user views their homepage on a placement platform.

FIG. 2B illustrates an example of a user interface with a match indicator presented to an administrator of a program in an admin dashboard.

FIG. 3 illustrates an example of a set of history data pertaining to a set of prior users of a placement platform in one or more embodiments.

FIG. 4 illustrates a data matcher in one or more embodiments of a placement platform with match indicators.

FIGS. 5-7 are examples of how a data matcher can match a current user to a particular program for example sets of current data pertaining to a current user.

FIGS. 8A-8C show an example of how a placement platform can update its match indicators in response to each of a sequence of activities undertaken by a current user.

FIG. 9 illustrates an example cloud-based implementation of a placement platform with matching.

FIG. 10 illustrates a method for program placement with matching in one or more embodiments.

FIG. 11 illustrates a computing system upon which portions of a placement platform with matching can be implemented.

### DETAILED DESCRIPTION

Reference will now be made in detail to the various embodiments of the present disclosure, examples of which are illustrated in the accompanying drawings. Like elements in the various figures are denoted by like reference numerals for consistency. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, have not been described in detail so as not to unnecessarily obscure aspects of the present disclosure.

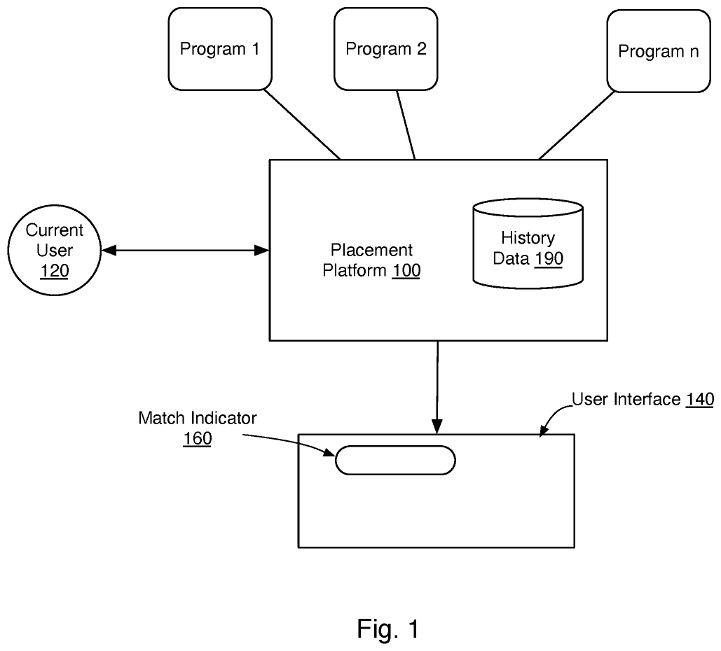

FIG. 1 illustrates a placement platform 100 in one or more embodiments. The placement platform 100 generates a user interface 140 that includes a match indicator 160 of how well a current user 120 of the placement platform 100 matches to a set of programs 1-n registered on the placement platform 100 based on a set of history data 190 pertaining to a set of prior users 1-m of the placement platform 100.

The match indicator 160 in the user interface 140 can include any combination of text, graphics, multimedia, etc., to convey a quality of a match between the current user 120 and one or more of the programs 1-n. For example, the match indicator 160 can indicate that the current user has a 90 percent chance of placing in program 1, or a very high chance of placing in program 1 using graphical match indicators, colors, etc.

In one or more embodiments, the user interface 140 is presented to the current user 120 while the current user 120 seeks placement among the programs 1-n by interacting with the placement platform 100. For example, the user interface 140 can enable the current user 120 to browse among the programs 1-n and view program summaries of each along with match indicators of how well the current user 120 matches to the programs 1-n depicted in the user interface 140.

In one or more embodiments, the user interface 140 is presented to administrators of the programs 1-n who seek to evaluate the current user 120. For example, the user interface 140 can be an administrator dashboard that enables an administrator to browse among a variety of current users registered on the placement platform 100 and view user profiles of each current user along with match indicators of how well each current user depicted in the dashboard matches to their program.

In one or more embodiments, the programs 1-n are medical residency programs, e.g. residency programs associated with medical schools or other institutions, and the current user 120 is a new medical school graduate seeking placement in a residency program. In the following examples, the programs 1-n include the Stanford Health Care Anesthesiology Program, the Brigham and Women's Hospital Anesthesiology Program, the Massachusetts General Hospital Anesthesiology Program, and the University of California San Francisco (UCSF) Anesthesiology Program.

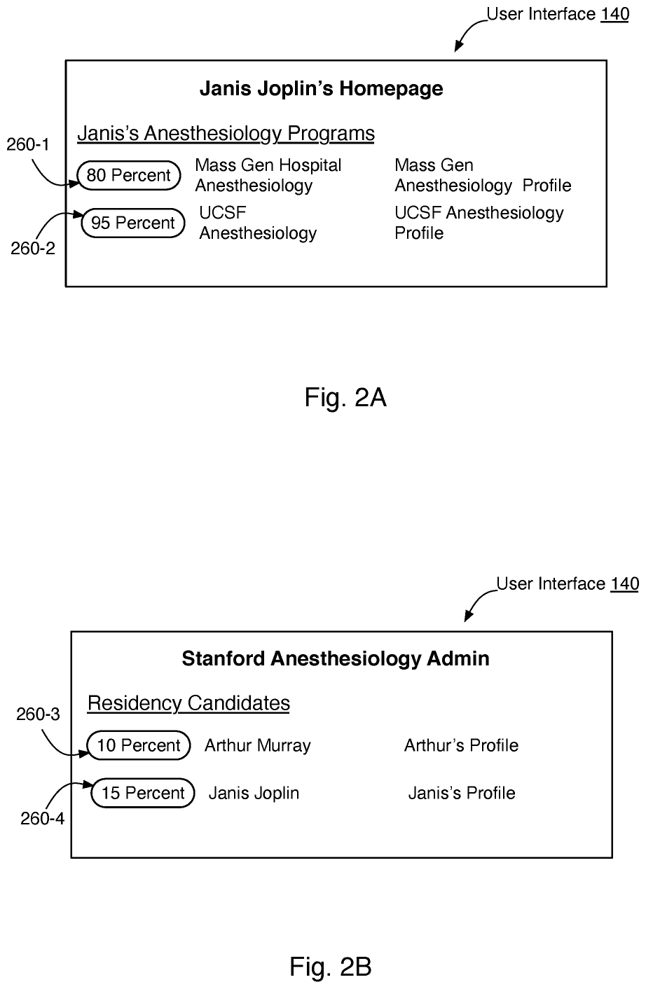

FIG. 2A illustrates an example of the user interface 140 presented to the current user 120, Janis Joplin, while she views her homepage on the placement platform 100. In one or more embodiments, the user interface 140 enables Janis to browse and view profiles of the programs 1-n. In this example, Janis views the Massachusetts General Hospital Anesthesiology Program and the UCSF Anesthesiology Program, and views respective match indicators 260-1 and 260-2. The match indicator 260-1 indicates that Janis has an 80 percent quality of match with the Massachusetts General Hospital Anesthesiology Program and the match indicator 260-2 indicates Janis has a 95 percent quality of match with the UCSF Anesthesiology Program.

FIG. 2B illustrates an example of the user interface 140 presented to an administrator of one of the programs 1-n. In this example, the user interface 140 is an administrator dashboard for the Stanford Health Care Anesthesiology Program. The administrator dashboard enables the Stanford Health Care administrator to browse and view profiles of the current users of the placement platform, in this example Arthur Murray and Janis Joplin, and view respective match indicators 260-3 and 260-4. The match indicator 260-3 indicates that Arthur has a 10 percent quality of match with the Stanford Health Care Anesthesiology Program and the match indicator 260-4 indicates Janis has a 15 percent quality of match with the Stanford Health Care Anesthesiology Program.

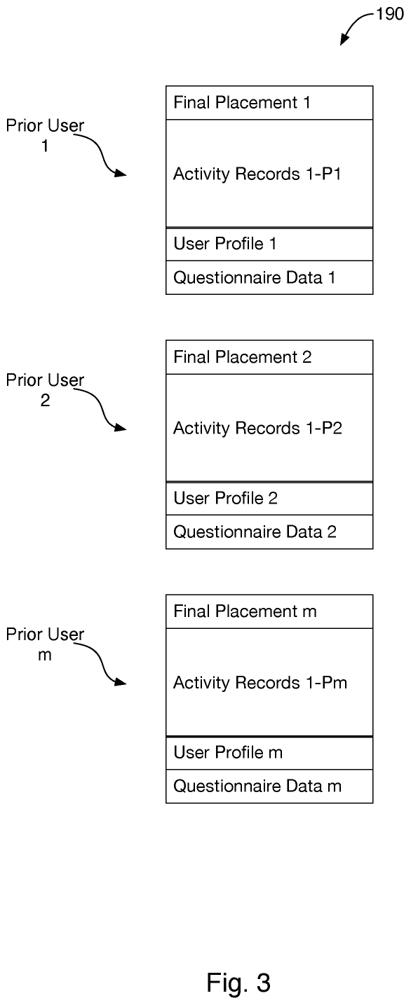

FIG. 3 illustrates an example of the history data 190 in one or more embodiments. The history data 190 includes a respective set of history data for each of the prior users 1-m of the placement platform 100. Each set of history data for the prior users 1-m includes a respective final placement 1-m which specifies in which of the programs 1-n the respective prior user 1-m placed, a respective set of activity records 1-P1 through 1-Pm describing a set of activities undertaken by the respective prior user 1-m in reaching the respective final placement 1-m, a respective user profile 1-m of the respective prior user 1-m, and a respective set of questionnaire data 1-m of the respective prior user 1-m.

Examples of activities logged in the activity records 1-P1 through 1-Pm include initiating a placement process with a specified one of the programs 1-n, receiving an invitation from a specified one of the programs 1-n to schedule an interview, scheduling an interview with a specified one of the programs 1-n, cancelling an interview with a specified one of the programs 1-n, rescheduling an interview with a specified one of the programs 1-n, being placed on a waitlist for a specified one of the programs 1-n etc. The activity records 1-P1 through 1-Pm can include parameters for the respective activities, e.g., date and time parameters.

Examples of the prior user profiles 1-m include demographic data, e.g., age, gender, race, place of birth, etc., educational data, e.g., medical school attended, relevant test scores, etc., extracurricular activities, awards, etc.

The questionnaire data 1-m specifies answers to specific questions presented to the respective prior user 1-m by the programs 1-n. In one or more embodiments, the placement platform 100 provides administrators of the programs 1-n with a mechanism for presenting questionnaires to the users of the placement platform 100. The questionnaires can be tailored to the specific needs of the programs 1-n. For example, if the Massachusetts General Hospital Anesthesiology Program seeks candidates who are left-handed, or bilingual, or who speak a particular language, or who have had particular life experiences, that data can be acquired in a questionnaire and used as a basis for matching.

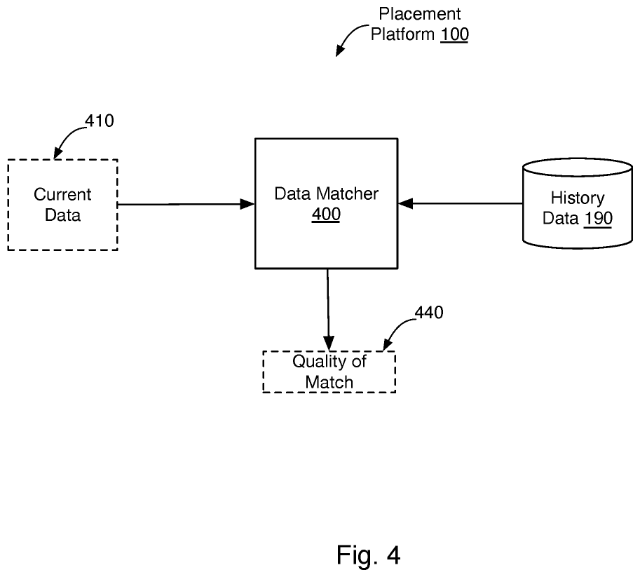

FIG. 4 illustrates a data matcher 400 in one or more embodiments of the placement platform 100. The data matcher 400 determines a quality of match 440 between the current user 120 and the programs 1-n by matching a set current data 410 pertaining to the current user 120 to analogous relevant portions of the history data 190. For example, the quality of match 440 can specify the odds, e.g., 70 percent, 80 percent, etc., that the current user 120 will end up placing in the Massachusetts General Hospital Anesthesiology Program or the UCSF Anesthesiology Program, etc.

The current data 410 can include data associated with one or more activities undertaken by the current user 120 when seeking placement among the programs 1-n. Examples of activities of the current user 120 include initiating a placement process with a specified one of the programs 1-n, receiving an invitation from a specified one of the programs 1-n, scheduling an interview with a specified one of the programs 1-n, cancelling an interview with a specified one of the programs 1-n, rescheduling an interview with a specified one of the programs 1-n, being placed on a waitlist for a specified one of the programs 1-n etc. The current data 410 can include parameters for the respective activities, e.g., date and time parameters.

The current data 410 can include a user profile of the current user 120, e.g., age, gender, race, place of birth, etc., educational data, e.g., medical school attended, relevant test scores, etc., extracurricular activities, awards, etc. The current data 410 can include questionnaire data obtained from the current user 120 by one or more of the programs 1-n.

The data matcher 400 can determine the quality of match 440 based on any combination, aggregation, characterization, statistical analysis, etc., of the information in the current data 410. For example, the data matcher 400 can determine the quality of match 440 based on one or more aspects of the user profile for the current user 120, based on any activity or sequence of activities, or aggregations of activities specified in the current data 410, or any combination of user profile data, questionnaire data, and activity data contained in the current data 410.

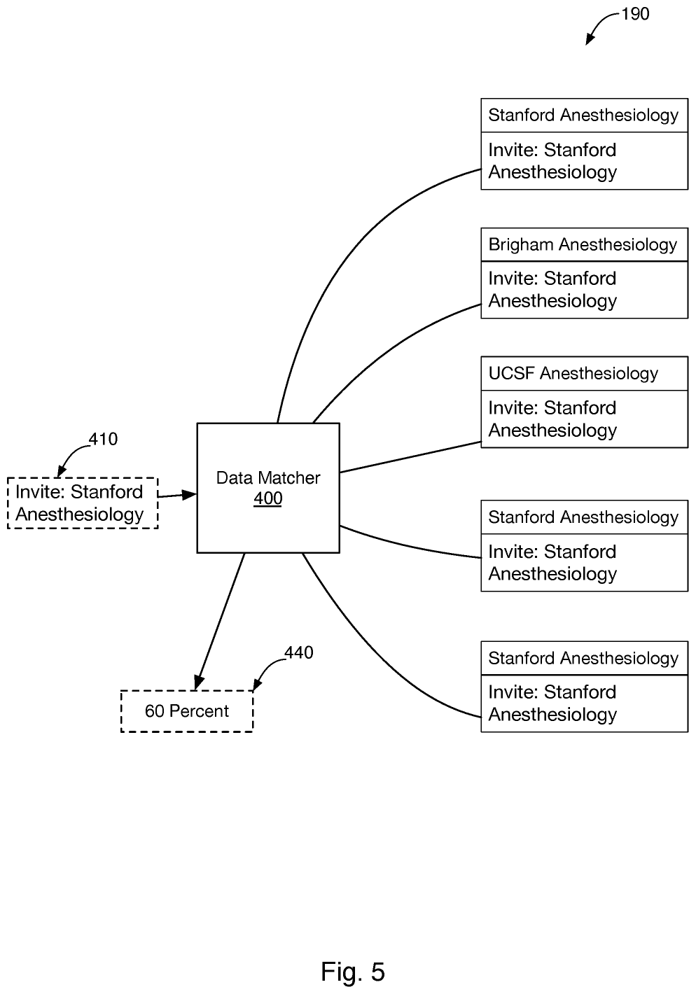

FIG. 5 illustrates an example of how the data matcher 400 can determine the quality of match 440 between the current user 120 and the Stanford Health Care Anesthesiology Program when the current data 410 specifies that the current user 120 has received an invite from the Stanford Health Care Anesthesiology Program. The data matcher 400 in this example searches the history data 190 and finds that five of the prior users 1-m received invites from the Stanford Health Care Anesthesiology Program and three of those five ended up placing in the Stanford Health Care Anesthesiology Program, thereby yielding the quality of match 440 of 60 percent.

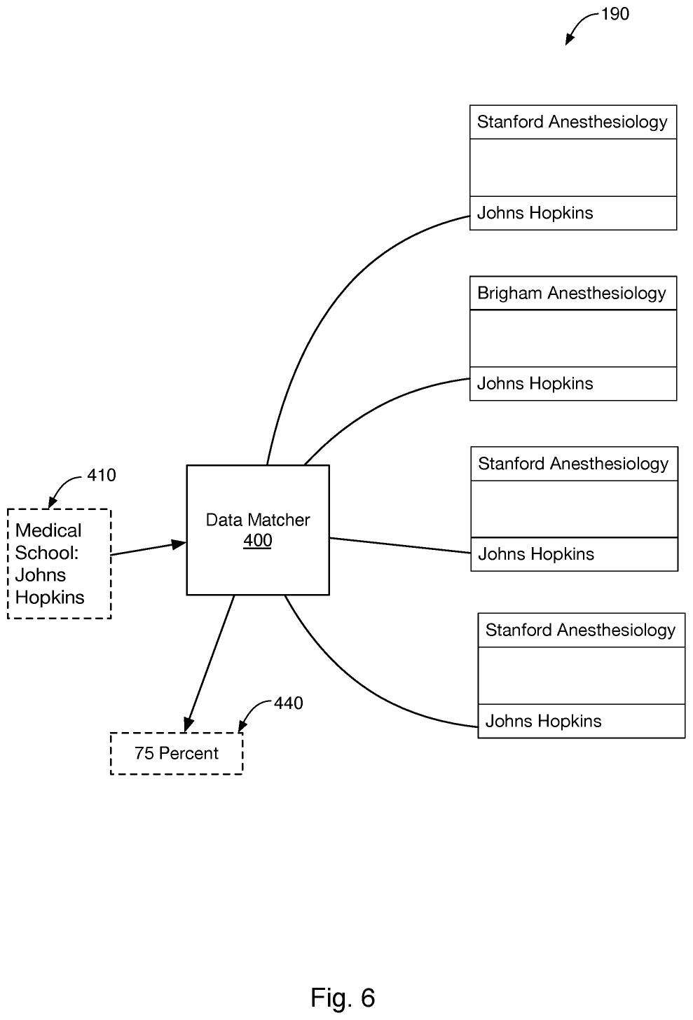

FIG. 6 illustrates an example of how the data matcher 400 can determine the quality of match 440 between the current user 120 and the Stanford Health Care Anesthesiology Program when the current data 410 specifies that the current user 120 attended Johns Hopkins medical school. The data matcher 400 in this example searches the history data 190 and finds that four of the prior users 1-m graduated Johns Hopkins and three of them ended up placing in the Stanford Health Care Anesthesiology Program, thereby yielding the quality of match 440 of 75 percent.

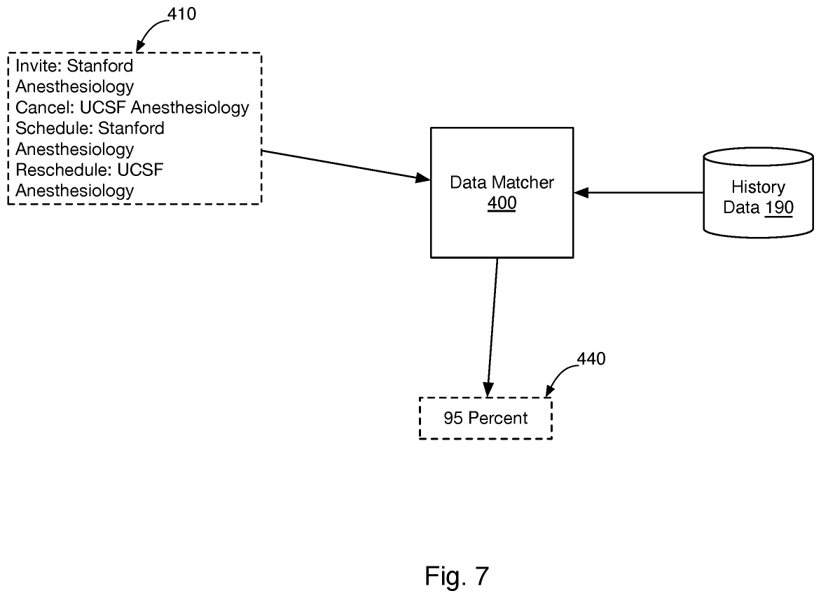

FIG. 7 illustrates an example of how the data matcher 400 can determine the quality of match 440 between the current user 120 and the Stanford Health Care Anesthesiology Program when the current data 410 specifies that the current user 120 received an invite from the Stanford Health Care Anesthesiology Program, then cancelled an interview at the UCSF Anesthesiology Program, then scheduled at the Stanford Health Care Anesthesiology Program, and then rescheduled the UCSF Anesthesiology Program. The data matcher 400 in this example searches the activity records 1-P1 through 1-Pm for the prior users 1-m that engaged in that sequence of activities and determines how many of those matching prior users 1-m ended up placing in the Stanford Health Care Anesthesiology Program.

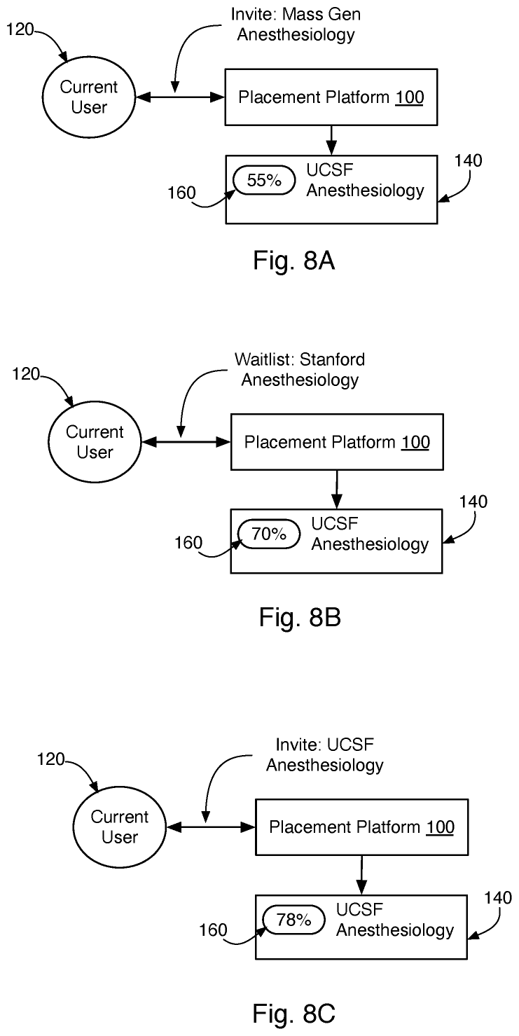

FIGS. 8A-8C illustrate how the placement platform 100 updates the match indicator 160 in the user interface 140 in response to each of a sequence of activities in which the current user 120 receives an invite from the Massachusetts General Hospital Anesthesiology Program, and then is placed on a waitlist for the Stanford Health Care Anesthesiology Program, and then receives an invite from the UCSF Anesthesiology Program. The match indicator 160 in this example depicts the changing likelihoods that the current user 120 will place in the UCSF Anesthesiology Program as the data matcher 400 updates the likelihood of ending up at the UCSF Anesthesiology Program for each new piece of activity data of the current user 120.

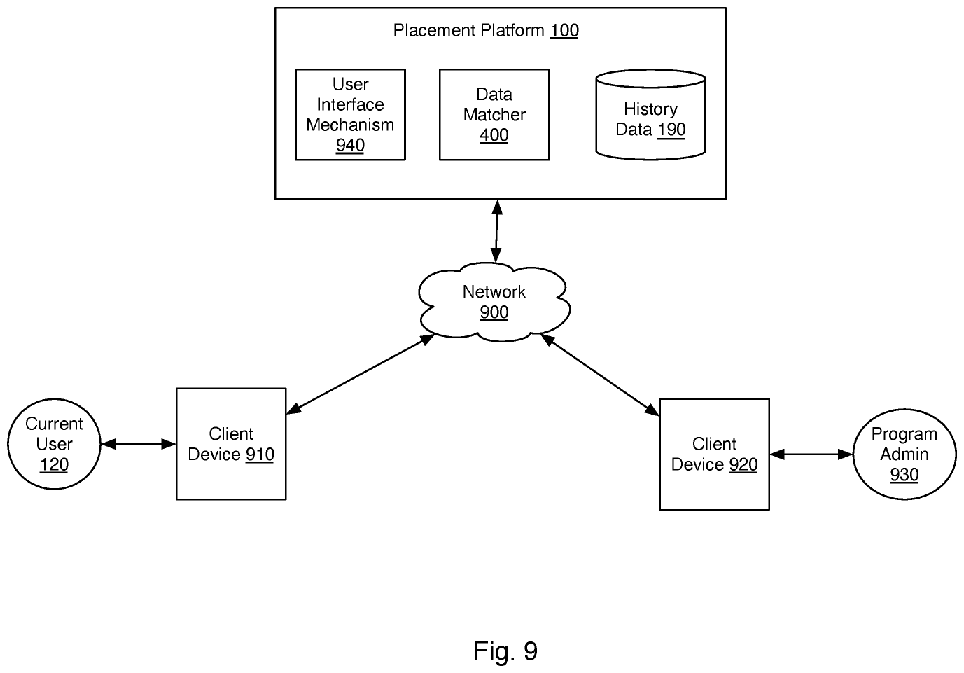

FIG. 9 illustrates an example cloud-based implementation of the placement platform 100 in which the current user 120 and a program admin 930 of one of the programs 1-n access the placement platform 100 via a network 900 using, e.g., internet protocols, via respective client devices 910 and 920. The client devices 910 and 920 can be mobile devices, desktop computers, etc. The placement platform 100 includes a user interface mechanism 940 that generates user interfaces, e.g., home pages, dashboards, etc., accessed by the current user 120 and the program admin 930, including match indicators of the likelihoods of placement as disclosed above.

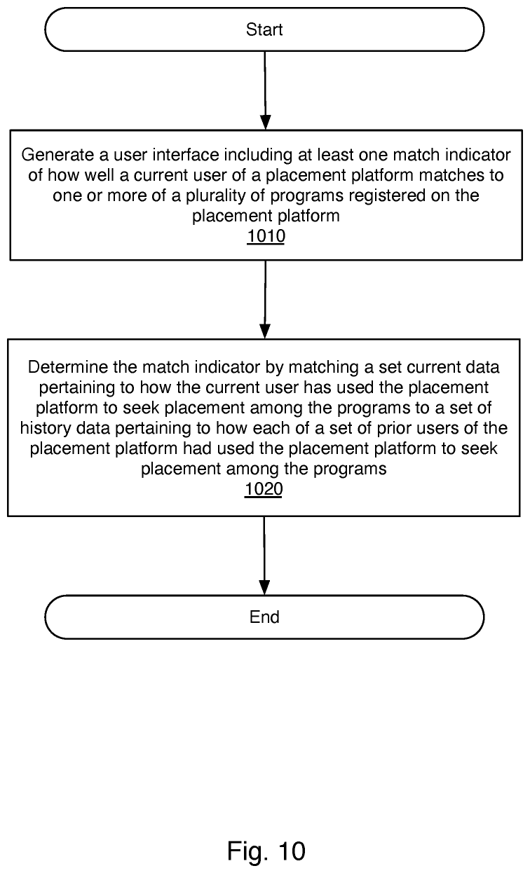

FIG. 10 illustrates a method for program placement with matching in one or more embodiments. While the various steps in this flowchart are presented and described sequentially, one of ordinary skill will appreciate that some or all of the steps can be executed in different orders and some or all of the steps can be executed in parallel. Further, in one or more embodiments, one or more of the steps described below can be omitted, repeated, and/or performed in a different order. Accordingly, the specific arrangement of steps shown in FIG. 10 should not be construed as limiting the scope of the invention.

At step 1010, a user interface is generated including at least one match indicator of how well a current user of a placement platform matches to one or more of a plurality of programs registered on the placement platform. The user interface can be implemented in a home page of the current user of the placement platform or in an administrator dashboard of any of the administrators of the programs registered on the placement platform.

At step 1020, the match indicator in the user interface is determined by matching a set current data pertaining to how the current user has used the placement platform to seek placement among the programs to a set of history data pertaining to how each of a set of prior users of the placement platform had used the placement platform to seek placement among the programs. For example, the likelihood that the current user will place in a program depicted in the user interface can be determined in response to the current data pertaining to the current user and the history data and then the match indicator can be adapted to depict that likelihood.

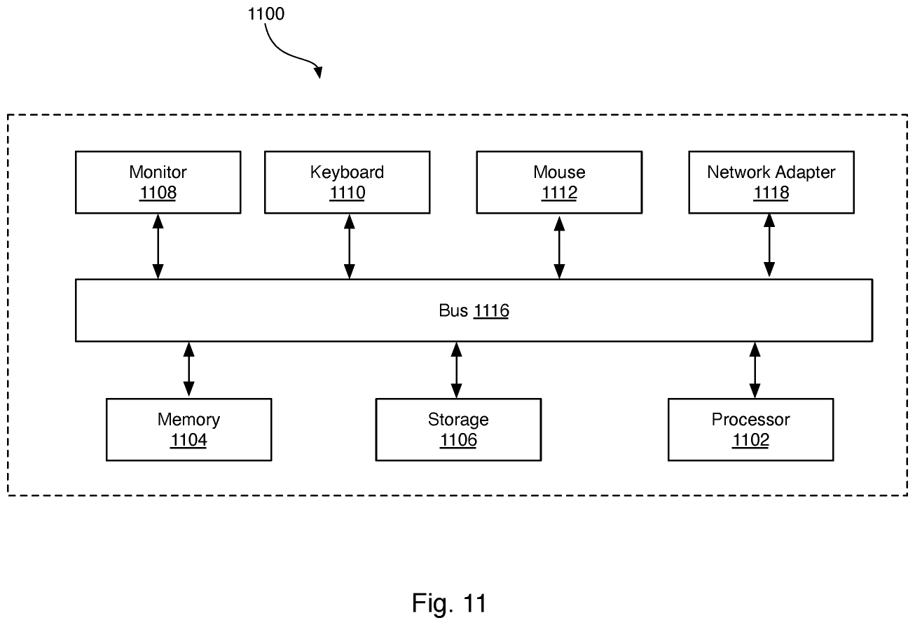

FIG. 11 illustrates a computing system 1100 upon which portions of the placement platform 100 can be implemented. The computing system 1100 includes one or more computer processor(s) 1102, associated memory 1104 (e.g., random access memory (RAM), cache memory, flash memory, etc.), one or more storage device(s) 1106 (e.g., a hard disk, an optical drive such as a compact disk (CD) drive or digital versatile disk (DVD) drive, a flash memory stick, etc.), a bus 1116, and numerous other elements and functionalities.

The computer processor(s) 1102 may be an integrated circuit for processing instructions. For example, the computer processor(s) may be one or more cores or micro-cores of a processor. The computing system 1100 may also include one or more input device(s), e.g., a touchscreen, keyboard 1110, mouse 1112, microphone, touchpad, electronic pen, or any other type of input device. Further, the computing system 1100 may include one or more monitor device(s) 1108, such as a screen (e.g., a liquid crystal display (LCD), a plasma display, touchscreen, cathode ray tube (CRT) monitor, projector, or other display device), external storage, input for an electric instrument, or any other output device. The computing system 1100 may be connected to, e.g., a local area network (LAN), a wide area network (WAN) such as the Internet, mobile network, or any other type of network) via a network adapter 1118.

While the foregoing disclosure sets forth various embodiments using specific diagrams, flowcharts, and examples, each diagram component, flowchart step, operation, and/or component described and/or illustrated herein may be implemented, individually and/or collectively, using a range of processes and components.

The process parameters and sequence of steps described and/or illustrated herein are given by way of example only. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While the invention has been described with respect to a limited number of embodiments, those skilled in the art, having benefit of this disclosure, will appreciate that other embodiments may be devised which do not depart from the scope of the invention as disclosed herein.

1. A placement platform, comprising:
a user interface including at least one match indicator of how well a current user of the placement platform matches to one or more of a plurality of programs registered on the placement platform; and
a data matcher that determines the match indicator by matching a set current data describing a set of placement activities undertaken by the current user via the placement platform while the current user is currently seeking placement among the programs to a set of history data describing a set of placement activities undertaken by a set of prior users of the placement platform back when the prior users were using the placement platform to seek placement among the programs.

2. The placement platform of claim 1, wherein the user interface is presented to the current user while the current user seeks placement among the programs.

3. The placement platform of claim 1, wherein the user interface is presented to an administrator of one of the programs who seeks to evaluate the current user.

4. The placement platform of claim 1, wherein the data matcher matches an aspect of a user profile of the current user to a corresponding aspect of a user profile of each of the prior users from the history data.

5. The placement platform of claim 1, wherein the data matcher matches a record in the current data of one or more of the placement activities undertaken by the current user via the placement platform to a set of records in the history data of one or more of the placement activities undertaken by each of the prior users.

6. The placement platform of claim 5, wherein the placement activities comprise at least one scheduling interaction with at least one of the programs.

7. The placement platform of claim 5, wherein the placement activities comprise a sequence of scheduling interactions with at least one of the programs.

8. The placement platform of claim 1, wherein the data matcher matches a record in the current data of one or more of the placement activities undertaken by the current user via the scheduling platform to a set of records in the history data of one or more of the placement activities undertaken by each of the prior users and further matches an aspect of a user profile of the current user to a corresponding aspect of a user profile of each of the prior users from the history data.

9. The placement platform of claim 1, wherein the data matcher updates the match indicator in response to a new placement activity undertaken by the current user via the placement platform.

10. The placement platform of claim 1, wherein the data matcher matches a set of questionnaire data obtained from the current user to a respective relevant set of questionnaire data in the history data obtained from each of the prior users.

11. A method for program placement, comprising:
generating a user interface including at least one match indicator of how well a current user of a placement platform matches to one or more of a plurality of programs registered on the placement platform; and
determining the match indicator by matching a set current data describing a set of placement activities undertaken by the current user via the placement platform while the current user is currently seeking placement among the programs to a set of history data describing a set of placement activities undertaken by a set of prior users of the placement platform back when the prior users were using the placement platform to seek placement among the programs.

12. The method of claim 11, further comprising presenting the user interface to the current user while the current user seeks placement among the programs.

13. The method of claim 11, further comprising presenting the user interface to an administrator of one of the programs who seeks to evaluate the current user.

14. The method of claim 11, wherein matching comprises matching an aspect of a user profile of the current user to a corresponding aspect of a user profile of each of the prior users from the history data.

15. The method of claim 11, wherein matching comprises matching a record in the current data of one or more of the placement activities undertaken by the current user via the placement platform to a set of records in the history data of one or more of the placement activities undertaken by each of the prior users.

16. The method of claim 15, wherein the placement activities comprise at least one scheduling interaction with at least one of the programs.

17. The method of claim 15, wherein the placement activities comprise a sequence of scheduling interactions with at least one of the programs.

18. The method of claim 11, wherein matching comprises matching a record in the current data of one or more of the placement activities undertaken by the current user via the scheduling platform to a set of records in the history data of one or more of the placement activities undertaken by each of the prior users and further matching an aspect of a user profile of the current user to a corresponding aspect of a user profile of each of the prior users from the history data.

19. The method of claim 11, further comprising updating the match indicator in response to a new placement activity undertaken by the current user via the placement platform.

20. The method of claim 11, wherein matching comprises matching a set of questionnaire data obtained from the current user to a respective relevant set of questionnaire data in the history data obtained from each of the prior users.
